(19) United States
(12) Patent Application Publication
(10) Pub. No.: US 2017/0151957 A1
(43) Pub. Date: Jun. 1, 2017

(54) Vehicle with interactions with wearable device to provide health or physical monitoring

(71) Applicant: BRAGI GmbH, Munchen (DE)

(72) Inventor: Peter Vincent Boesen, Munchen (DE)

(21) Appl. No.: 15/357,012

(22) Filed: Nov. 21, 2016

Related U.S. Application Data
(60) Provisional application No. 62/260,444, filed on Nov. 27, 2015.

(51) Int. Cl. B60W 40/08; A61B 5/1455; A61B 5/01; A61B 5/145; A61B 5/00

(52) U.S. Cl. CPC B60W 40/08; A61B 5/14532; A61B 5/4845; A61B 5/01; A61B 5/14551; A61B 5/747; A61B 5/6803; B60W 2040/0872; B60W 2540/22; B60W 2540/24; B60W 2540/26

(57) ABSTRACT

A system includes a vehicle, a vehicle network disposed within the vehicle, and an earpiece having an earpiece housing, a physiological monitoring sensor, an intelligent control system operatively connected to the physiological monitoring sensor and disposed within the ear piece housing, and a wireless transceiver disposed within the earpiece housing and operatively connected to the intelligent control system. The vehicle is configured to receive health data from the ear piece. A method includes sensing physiological data at one or more physiological sensors of an ear piece of an occupant of a vehicle, wirelessly communicating a representation of the physiological data from the ear piece to a vehicle network of the vehicle, and performing an action by the vehicle in response the physiological data to enhance safety of the vehicle.

### PRIORITY STATEMENT

This application claims priority to U.S. Provisional Patent Application 62/260,444, filed on Nov. 27, 2015, and entitled Vehicle with interactions with wearable device to provide health or physical monitoring, hereby incorporated by reference in its entirety.

### FIELD OF THE INVENTION

The present invention relates to wearable devices. More particularly, but not exclusively, the present invention relates to interactions between a vehicle and one or more wearable devices to provide health or physical monitoring of a driver or passenger of the vehicle.

### BACKGROUND

Vehicles may come with various types of electronics packages. These packages may be standard or optional and include electronics associated with improving safety. However, there are various problems and deficiencies with such offerings. What is needed are vehicles with improved electronics options which create, improve, or enhance safety or overall experience of vehicles. In particular, what is needed are vehicles which integrate with wearable devices.

### SUMMARY

Therefore, it is a primary object, feature, or advantage of the present invention to improve over the state of the art.

It is another object, feature, or advantage of the present invention to communicate between vehicle systems and wearable devices.

It is a further object, feature, or advantage of the present invention to use wearable devices within vehicles and to provide enhanced vehicle functionality.

It is another object, feature, or advantage of the present invention to enhance the safety of a vehicle using wearable devices.

One or more of these and or other objects, features, or advantages of the present invention will become apparent from the specification and claims that follow. No single embodiment need provide each and every object, feature, or advantage. Different embodiments may have different objects, features, or advantages. Therefore, the present invention is not to be limited to or by any objects, features, or advantages stated herein.

According to one aspect a system includes a vehicle, a vehicle network disposed within the vehicle, and an earpiece comprising an earpiece housing, a physiological monitoring sensor, a processor operatively connected to the physiological monitoring sensor and disposed within the ear piece housing, and a wireless transceiver disposed within the earpiece housing and operatively connected to the processor. The vehicle is configured to receive health data from the ear piece. The physiological monitoring sensor may include one or more of an inertial sensor, a glucose sensor, an alcohol sensor, a temperature sensor, and/or a pulse oximeter. The vehicle may determine the presence of a health condition based on the health data and performs an action to improve safety of the vehicle. The action may include actions such as disabling the vehicle, playing an audio message, placing a phone call, mapping a destination using a navigation system of the vehicle, adjusting an audio setting to increase volume, opening a window of the vehicle, and/or adjusting a temperature setting of the vehicle. The earpiece may determine presence of a health condition based on the health data and communicate an alert to the vehicle and the vehicle may perform an action to improve safety of the vehicle in response to the health condition.

According to another aspect a method may include sensing physiological data at one or more physiological sensors of an ear piece of an occupant of a vehicle, wirelessly communicating a representation of the physiological data from the ear piece to a vehicle network of the vehicle, and performing an action by the vehicle in response the physiological data to enhance safety of the vehicle. The physiological data may include pulse oximeter data, inertial sensor data, temperature data, glucose sensor data, and/or data from other types of sensors.

### BRIEF DESCRIPTION OF THE DRAWINGS

FIG. 1 illustrates one example of use of a eatable device in conjunction with a vehicle.

FIG. 2 illustrates a wearable device in the form of a set of ear pieces.

FIG. 3 is a block diagram illustrating a device.

FIG. 4 illustrates a system which includes ear pieces in communication with a vehicle.

FIG. 5 illustrates a wearable device in communication with various vehicle systems through a vehicle network.

### DETAILED DESCRIPTION

Some of the most important factors in selecting a vehicle such as car may be the technology available to enhance the experience. This may be of particular importance in certain vehicle segments such as for luxury vehicles. Another important factor in selecting a vehicle may be the available safety features. According to various aspects, the present invention allows for wearable devices such as earpieces to enhance the overall safety of the vehicle. Therefore, it is expected that the technology described herein will make any vehicle so equipped more desirable to customers, more satisfying to customers, and potentially more profitable for the vehicle manufacturer. Similarly at least some of the various aspects may be added to existing vehicles as after-market accessories to improve the safety or experience of existing vehicles,

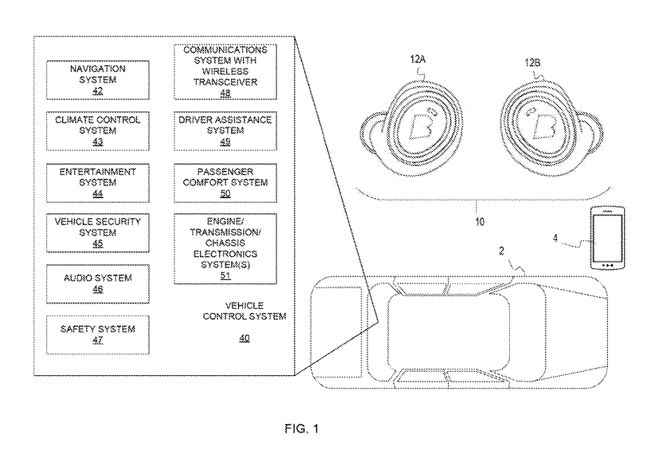

FIG. 1 illustrates one example of use of a wearable device in conjunction with a vehicle. A shown in FIG. 1 there is a vehicle 2. Although the vehicle shown is a full-size sedan, it is contemplated that the vehicle may be of any number of types of cars, trucks, sport utility vehicles, vans, mini-vans, automotive vehicles, commercial vehicles, agricultural vehicles, construction vehicles, specialty vehicles, recreational vehicles, buses, motorcycles, aircraft, boats, ships, yachts, spacecraft, or other types of vehicles. The vehicle may be gas-powered, diesel powered, electric, solar-powered, or human-powered. The vehicle may be actively operated by a driver or may be partially or completely autonomous or self-driving. The vehicle 2 may have a vehicle control system 40. The vehicle control system is a system which may include any number of mechanical and electromechanical subsystems. As shown in FIG. 1, such systems may include a navigation system 42, an entertainment system 44, a vehicle security system 45, an audio system 46, a safety system 47, a communications system 48 preferably with a wireless transceiver, a driver assistance system 49, a passenger comfort system 50, and an engine/transmission, chassis electronics system(s) 51. Of course, other examples of vehicle control sub-systems are contemplated. In addition, it is to be understood that there may be overlap between some of these different vehicle systems and the presence or absence of these vehicle systems as well as other vehicle systems may depend upon the type of vehicle, the type of fuel or propulsion system, the size of the vehicle, and other factors and variables. In the automotive context, examples of the driver assistance system 49 may include one or more subsystems such as a lane assist system, a speed assist system, a blind spot detection system, a park assist system, and an adaptive cruise control system. In the automotive context, examples of the passenger comfort system 50 may include one or more subsystems such as automatic climate control, electronic seat adjustment, automatic wipers, automatic headlamps, and automatic cooling. In the automotive context, examples of the safety system 47 may include active safety systems such as air bags, hill descent control, and an emergency brake assist system. Aspects of the navigation system 42, the entertainment system 44, the audio system 46, and the communications system 48 may be combined into an infotainment system.

One or more wearable devices such as a set of earpieces 10 including a left earpiece 12A and a right earpiece 1213 may in operative communication with the vehicle control system 40 such as through the communication system 48. For example, the communication system 48 may, provide a Bluetooth or BLE link to wearable devices or may otherwise provide for communications with the wearable devices preferably through wireless communications. The vehicle 2 may communicate with the wearable device(s) directly, or alternatively, or in addition, the vehicle 2 may communicate with the wearable device(s) through an intermediary device such as a mobile device 4 which may be a mobile phone, a tablet, or other type of mobile device.

As will be explained in further details with respect to various examples, the wearable device(s) 10 interact with the vehicle control system 40 in any number of different ways. For example, the wearable device(s) 10 may provide sensor data, identity information, stored information, streamed information, or other types of information to the vehicle. Based on this information, the vehicle may take any number of actions which may include one or more actions taken by the vehicle control system (or subsystems thereof). In addition, the vehicle 2 may communicate sensor data, identity infer nation, stored information, streamed information or other types of information to the wearable device(s) 10.

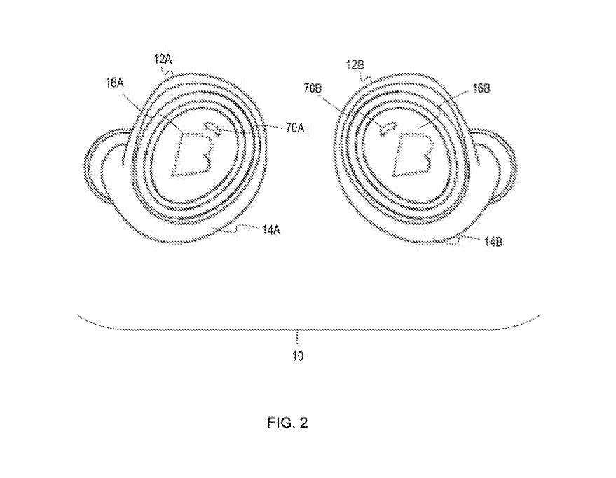

FIG. 2 illustrates one example of a wearable device in the form of a set of ear pieces 10 in greater detail. FIG. 1 illustrates a set of earpiece wearables 10 which includes a left earpiece 12A and a right earpiece 12B. Each of the earpieces wearables 12A, 12B has an earpiece wearable housing 14A, 14B which may be in the form of a protective shell or casing and may be an in-the-ear earpiece housing. A left infrared through ultraviolet spectrometer I6A and right infrared through ultraviolet spectrometer 16B is also shown. Each earpiece 12A, 12B may include one or more microphones 70A, 70B. Note that the air microphones 70A, 70B are outward facing such that the air microphones 70A, 70B may capture ambient environmental sound. It is to be understood that any number of microphones may be present including air is conduction microphones, bone conduction microphones, or other audio sensors.

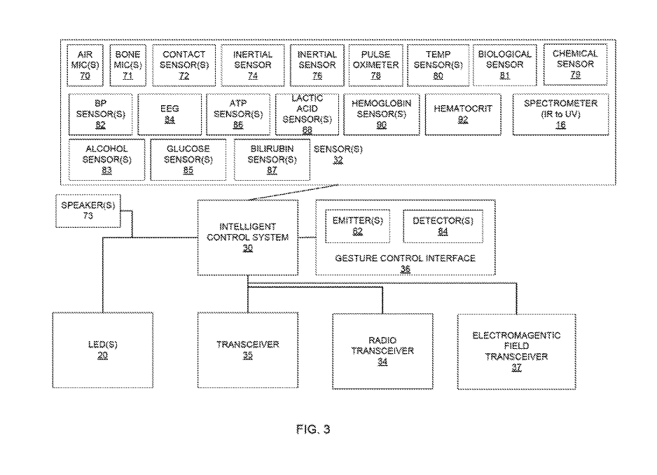

FIG. 3 is a block diagram illustrating a device. The device may include one or more LEDs 20 electrically connected to an intelligent control system 30. The intelligent control system 30 may include one or more processors, microcontrollers, application specific integrated circuits, or other types of integrated circuits. The intelligent control system 30 may also be electrically connected to one or more sensors 32. Where the device is an earpiece, the sensor(s) may include an inertial sensor 74, another inertial sensor 76. Each inertial sensor 74, 76 may include an accelerometer, a gyro sensor or gyrometer, a magnetometer or other type of inertial sensor. The sensor(s) 32 may also include one or more contact sensors 72, one or more bone conduction microphones 71, one or more air conduction microphones 70, one or more chemical sensors 79, a pulse oximeter 76, a temperature sensor 80, or other physiological or biological sensor(s). Further examples of physiological or biological sensors include an alcohol sensor 83, glucose sensor 85, or bilirubin sensor 87. Other examples of physiological or biological sensors may also be included in the device. These may include a blood pressure sensor 82, an electroencephalogram (EEG) 84, an Adenosine Triphosphate (ATP) sensor, a lactic acid sensor 88, a hemoglobin sensor 90, a hematocrit sensor 92 or other biological or chemical sensor.

A spectrometer 16 is also shown. The spectrometer 16 may be an infrared (IR) through ultraviolet (UV) spectrometer although it is contemplated that any number of wavelengths in the infrared, visible, or ultraviolet spectrums may be detected. The spectrometer 16 is preferably adapted to measure environmental wavelengths for analysis and recommendations and thus preferably is located on or at the external facing side of the device,

A gesture control interface 36 is also operatively connected to or integrated into the intelligent control system 30. The gesture control interface 36 may include one or more emitters 82 and one or more detectors 84 for sensing user gestures. The emitters may be of any number of types including infrared LEDs. The device may include a transceiver 35 which may allow for induction transmissions such as through near field magnetic induction. A short range transceiver 34 using Bluetooth, BLE, UWB, Wi-Fi or other means of radio communication may also be present. The short range transceiver 34 may he used to communicate with the vehicle control system. In operation, the intelligent control system 30 may be configured to convey different information using one or more of the LED(s) 20 based on context or mode of operation of the device. The various sensors 32, the processor 30, and other electronic components may be located on the printed circuit board of the device. One or more speakers 73 may also be operatively connected to the intelligent control system 30.

An electromagnetic (E/M) field transceiver 37 or other type of electromagnetic field receiver is also operatively connected to the intelligent control system 30 to link the processor 30 to the electromagnetic field of the user. The use of the E/M transceiver 37 allows the device to link electromagnetically into a personal area network or body area network or other device,

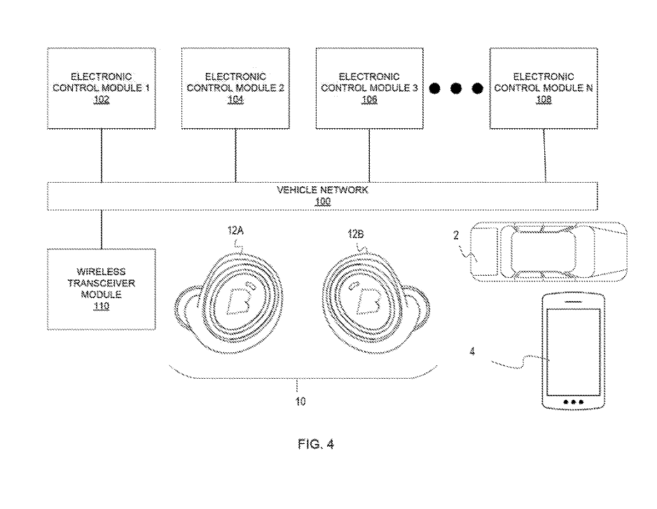

FIG. 4 illustrates another example of one or more wearable ear pieces in operative communication with a vehicle. In FIG. 4, a vehicle network 100 is shown. According to one aspect, the wearable devices 12A, 12B may communicate information through a vehicle network 100 associated with a vehicle 2. Data, instructions, alerts, or other information may be communicated over the vehicle network 100 or vehicle bus to and from the wearable devices. Protocols which are used may include a Controller Area Network (CAN), Local interconnect Network (LIN), or others including proprietary network protocols or network protocol overlays,

Various types of electronic control modules 102, 104, 106, 108 or electronic control units may communicate over the network. 100 of the vehicle. These may include electronic modules such as an engine control unit (ECU), a transmission control unit (TCU), an anti-lock braking system (ABS), a body control module (BCM), a door control unit (DCU), an electric power steering control unit (PSCU), a human-machine interface (HM1), powertrain control module (PCM), speed control unit (SCU), telematic control unit (TCU), brake control unit (BCM), battery management system, vehicle navigation system, entertainment system, infotainment system, and numerous others. Any number of electronic control modules may be operatively connected to the vehicle network 100.

In one embodiment a wireless transceiver module 110 is operatively connected to a vehicle network 100 and it is the wireless transceiver module 110 which is in operative communication with one or more wearable devices such as wearable ear piece 12A, 12B.

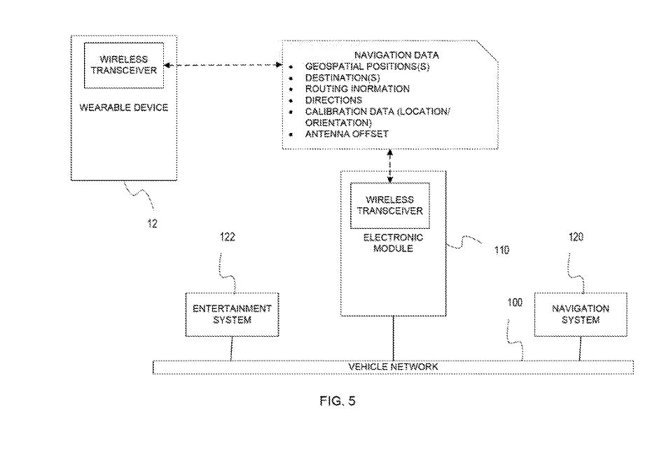

As shown in FIG. 5, one or more wearable devices 12 (including one or more ear pieces, from one or more different vehicle occupants) may communicate with a navigation system 120 of a vehicle. Although the communication may be performed directly between one or more systems of the vehicle and one or more ear pieces 12, in one embodiment a wireless transceiver module 110 may be operatively connected to the wearable ear piece 12 after the transceiver module 110 connects with or forms a wireless linkage with one or more of the ear pieces 12. The wireless transceiver module 110 may use any number of different types of communications and protocols including Bluetooth, Bluetooth Low Energy (BLE), ultra-wideband, Wi-Fi, or otherwise. The vehicle network 100 may provide for communicating with any number of different modules or systems including a navigation system 120 and an entertainment system 122.

According to another aspect, one or more wearable devices may provide for health monitoring of an individual such as a driver or passenger of the vehicle. The wearable devices may have any number of different sensors which ma be used for monitoring the health of an individual or other physical parameters of an individual. Examples of sensors may include one or more inertial sensors such as an accelerometer, a gyro sensor or gyrometer, a magnetometer or other type of inertial sensor. As shown in FIG. 3, the sensor(s) 32 may also include one or more contact sensors 72, one or more bone conduction microphones 71, one or more air conduction microphones 70, one or more chemical sensors 79, a pulse oximeter 78, a temperature sensor 80, or other physiological or biological sensor(s). Further examples of physiological or biological sensors include an alcohol sensor 83, glucose sensor 85, or bilirubin sensor 87. Other examples of physiological or biological sensors may also be included in the device. These may include a blood pressure sensor 82, an electroencephalogram (EEG) 84, an Adenosine Triphosphate (ATP) sensor, a lactic acid sensor 88, a hemoglobin sensor 90, a hematocrit sensor 92 or other biological or chemical sensor.

These various sensors may be used in any number of ways to provide feedback to the vehicle. For example, where the wearable device is an, earpiece, the inertial sensors may be used to track head movement of the driver. If the head movement of the driver is indicative that the user is falling asleep, such as downward movement of the chin and then snapping back of the head as the user catches themselves falling asleep, or other movements associated with a user falling asleep, then the earpiece may communicate a message to the vehicle. Upon receipt of the message, the vehicle may take any number of relevant, actions. This may include, turning on loud music, opening one or more windows, adjusting environmental controls such as making the cabin temperature cooler, locating the nearest rest stop or hotel or motel and providing navigation directions to it, turning on emergency hazard lights, disabling the vehicle, providing is one or more audio warnings, placing a phone call or any number of other actions.

Another example of use of a sensor is use of a glucose sensor. If the blood sugar of an individual is low as measured with a wearable device, the wearable device may communicate a message to the vehicle. Upon receipt of the message, the vehicle may take any number of relevant actions. This may include locating the nearest rest stop, restaurant, or gas station so that the individual may obtain something to eat, provide an audio message such as reminding the user to eat something, alert passengers within the vehicle or any number of other actions.

Another example of use of a sensor is use of an alcohol sensor. If the wearable device detects that the driver may be impaired based upon alcohol levels, then the wearable device may communicate an appropriate message to the vehicle which may disable its operation, provide an audio message, make a phone call, or perform any number of other actions.

Yet another example of use of a sensor is a pulse oximeter. If the wearable device detects that the driver heart rate of the driver is increasing then appropriate action may be taken. The vehicle, in this example, may combine the heart rate with other information. For example, the vehicle may determine that it is currently within a construction zone and that based on the heart rate, the driver may be frustrated. The vehicle may then respond in various ways such as by playing relaxing music or offering to play relaxing music, suggesting an alternative route or destination to avoid additional construction or traffic congestion, or other alternatives based on user history or preferences.

The various sensors may be used in any number of other ways including detecting health status or predicting health status which may be indicative of a health condition or event which may impair safe driving.

Returning to FIG. 5, various types of health data may be communicated to the vehicle including, without limitation, head movement, glucose levels, heart rate, and body temperature. In addition, one or more alert conditions may be communicated to the vehicle as well. Based on the health data and/or the alert condition(s) the vehicle may then perform the appropriate action in response to the condition as previously described.

Various methods, system, and apparatus have been shown and described relating to vehicles with wearable integration or communication. The present invention is not to be limited to these specific examples but contemplates any number of related methods, system, and is apparatus and these examples may vary based on the specific type of vehicle, the specific type of wearable device, the various types of health conditions and health data, the alert conditions where present, and the actions taken in response to health data and other considerations.

What is claimed is:

1. A system comprising:
a vehicle;
a vehicle network disposed within the vehicle;
an earpiece comprising an earpiece housing, a physiological monitoring sensor, an intelligent control system operatively connected to the physiological monitoring sensor and disposed within the ear piece housing, and a wireless transceiver disposed within the earpiece housing and operatively connected to the intelligent control system;
wherein the vehicle is configured to receive health data from the ear piece.

2. The system of claim 1 wherein the physiological monitoring sensor is an inertial sensor.

3. The system of claim 1 wherein the physiological monitoring sensor is a glucose sensor.

4. The system of claim 1 wherein the physiological monitoring sensor is an alcohol sensor.

5. The system of claim 1 wherein the physiological monitoring sensor is a temperature sensor.

6. The system of claim 1 wherein the physiological monitoring sensor is a pulse oximeter.

7. The system of claim 1 wherein the vehicle determines presence of a health condition based on the health data and performs an action to improve safety of the vehicle.

8. The system of claim 7 wherein the action comprises disabling the vehicle.

9. The system of claim 7 wherein the action comprises playing an audio message.

10. The system of claim 7 wherein the action comprises placing a phone call.

11. The system of claim 7 wherein the action comprises mapping a destination using a navigation system of the vehicle.

12. The system of claim 7 wherein the action comprises adjusting an audio setting to increase volume.

13. The system of claim 7 wherein the action comprises opening a window of the vehicle.

14. The system of claim 7 wherein the action comprises adjusting a temperature setting of the vehicle.

15. The system of claim 1 wherein the earpiece determines presence of a health condition based on the health data and communicates an alert to the vehicle.

16. The system of claim 15 wherein the vehicle performs an action to, improve safety of the vehicle in response to the health condition.

17. A method comprising:
sensing physiological data at one or more physiological sensors of an ear piece of an occupant of a vehicle;
wirelessly communicating a representation of the physiological data from the ear piece to a vehicle network of the vehicle;
performing an action by the vehicle in response the physiological data to enhance safety of the vehicle.

18. The method of claim 17 wherein the physiological data comprises pulse oximeter data.

19. The method of claim 18 wherein the physiological data further comprises inertial sensor data.

20. The method of claim wherein the physiological data further comprises temperature data.
